(19) United States
(12) Patent Application Publication
(10) Pub. No.: US 2025/0251378 A1
(43) Pub. Date: Aug. 7, 2025

(54) MULTI-SENSOR-BASED BATTERY ELECTRODE SCANNING FOR IN-PROCESS MULTI-LEVEL DEFECT DETECTION AND CLASSIFICATION

(71) Applicant: GM GLOBAL TECHNOLOGY OPERATIONS LLC, Detroit, MI (US)

(72) Inventors: Changbai TAN, Ann Arbor, MI (US); Robin JAMES, Rochester Hills, MI (US); Wayne CAI, Troy, MI (US); Raghavendra Saikrishna Keerthi MANDA, Northville, MI (US); Jorge F. ARINEZ, Rochester Hills, MI (US)

(21) Appl. No.: 18/429,632

(22) Filed: Feb. 1, 2024

(51) Int. Cl. G01N 33/00

(52) U.S. Cl. CPC G01N 33/00; G01N 33/0078

(57) ABSTRACT

A defect detection system includes: sensors of different types configured to scan battery electrode material and generate output signals including respective image data; a feature extractor module receives the output signals and performs feature extraction to fuse the image data of the output signals to generate feature maps for respective portions of the battery electrode material; a coarse detector, based on the feature maps, determines whether there are defects in the portions of the battery electrode material, and generates binary information for each of the portions indicating whether the portions include one or more defects; and a fine detector, based on the binary information and at least one of the feature maps and image data, classifies the defects. A control module performs one or more operations based on the detected and classified defects.

### INTRODUCTION

The information provided in this section is for the purpose of generally presenting the context of the disclosure. Work of the presently named inventors, to the extent it is described in this section, as well as aspects of the description that may not otherwise qualify as prior art at the time of filing, are neither expressly nor impliedly admitted as prior art against the present disclosure.

The present disclosure relates to defect detection systems for detecting defects in battery electrode material.

Traditional vehicles including internal combustion engines (ICEs) also include low-voltage (e.g., a 12 V) batteries. The low-voltage (LV) batteries are used for starting the ICEs and for powering various loads in the vehicles. The LV batteries are charged via alternators that convert mechanical energy from the ICEs to electrical energy to charge the LV batteries.

Electric vehicles, such as fully electric vehicles including battery electric vehicles (BEVs), and hybrid electric vehicles including plug-in hybrid electric vehicles (PHEVs), include high-voltage (HV) battery packs and LV batteries. A HV battery pack provides power to HV direct current (DC) loads and to an auxiliary power module that converts a high-voltage to a low-voltage to charge a low-voltage (LV) power source (or battery). The LV battery is used to power LV DC loads. The HV loads may include motors, which are used for propulsion purposes as well as other HV loads. The LV loads may include, for example, lights, window and seat motors, door locks, infotainment system devices, etc. A HV battery pack may have terminals at, for example, 400 V or 800 V. A LV battery may have terminals at, for example, 12V or 48V. A LV battery can be used to close a HV contactor when an electric vehicle is started to allow power from the HV battery pack to be supplied to motors and other HV loads.

### SUMMARY

A defect detection system is disclosed and includes: sensors of different types configured to scan battery electrode material and generate output signals including respective image data; a feature extractor module configured to receive the output signals and perform feature extraction to fuse the image data of the output signals to generate feature maps for respective portions of the battery electrode material; a coarse detector configured, based on the feature maps, to determine whether there are defects in the portions of the battery electrode material, and generate binary information for each of the portions indicating whether the portions include one or more defects; and a fine detector configured, based on the binary information and at least one of the feature maps and image data, to classify the defects. A control module is configured to at least one of i) report the classified defects, ii) determine whether one or more of the portions of the battery electrode material which have one or more defects ought to be discarded, iii) perform operations to discard the one or more portions of the battery electrode material which have one or more defects, and iv) perform feedback process control operations to prevent future occurrences defect similar to the classified defects.

In other features, the sensors include at least one surface scanning sensor, at least one interior scanning sensor, and at least one interface scanning sensor.

In other features, the at least one surface scanning sensor includes at least one of a camera, a laser 3D profiler, and a flash thermography scanner. The at least one interior scanning sensor comprises at least one of an X-ray scanner, a neutron imaging sensor, an eddy current sensor, and a beta gauging sensor. The at least one interface scanning sensor includes at least one of a terahertz scanner and an ultrasound sensor.

In other features, the coarse detector is configured to implement a first convolutional neural network and perform deep learning to determine whether there are defects in the portions of the battery electrode material. The fine detector is configured to implement a second convolutional neural network and perform deep learning to classify the defects.

In other features, the fine detector is configured, when classifying the defects, to determine whether each of the defects is a dark band defect, a contamination defect, a crack, an embrittlement defect, a wrinkle, an edge imperfection, a polka dot, a void, or a delamination.

In other features, the fine detector analyzes portions of the at least one of the feature maps and image data associated with the portions of the battery electrode material that include defects and does not analyze portions of the at least one of the feature maps and image data associated with the portions of the battery electrode material that do not include defects.

In other features, the control module is configured, prior to the coarse detector determining whether there are defects in portions of the battery electrode material, to perform a processing procedure on the image data received from the sensors. The processing procedure includes at least one of aligning the image data, normalizing the image data, synchronizing the image data, and cropping the image data to generate processed data. The control module is configured to generate fused data of the feature maps based on the processed data.

In other features, the processing procedure includes aligning the image data, normalizing the image data, synchronizing the image data, and cropping the image data.

In other features, the fine detector is configured to perform i) multi-classification to identify defect types, and ii) localization to locate and scale bounding boxes to defects present in an image.

In other features, the fine detector is configured to analyze portions of the at least one of the feature maps and image data containing at least one defect and to not analyze portions of the at least one of the feature maps and image data not containing a defect.

In other features, a defect detection method includes: scanning battery electrode material, via sensors of different types, and generating output signals including respective image data; receiving the output signals and performing feature extraction to fuse the image data of the output signals to generate feature maps for respective portions of the battery electrode material; based on the feature maps, determining whether there are defects in the portions of the battery electrode material, and generate binary information for each of the portions indicating whether the portions include one or more defects; and based on the binary information and at least one of the feature maps and image data, classifying the defects. The method further includes at least one of i) reporting the classified defects, ii) determining whether one or more of the portions of the battery electrode material which have one or more defects ought to be discarded, iii) performing operations to discard the one or more portions of the battery electrode material which have one or more defects, and iv) performing feedback process control operations to prevent future occurrences defect similar to the classified defects.

In other features, the sensors include at least one surface scanning sensor, at least one interior scanning sensor, and at least one interface scanning sensor.

In other features, the at least one surface scanning sensor includes at least one of a camera, a laser 3D profiler, and a flash thermography scanner. The at least one interior scanning sensor includes at least one of an X-ray scanner, a neutron imaging sensor, an eddy current sensor, and a beta gauging sensor. The at least one interface scanning sensor includes at least one of a terahertz scanner and an ultrasound sensor.

In other features, the defect detection method further includes: implementing a first convolutional neural network and performing deep learning to determine whether there are defects in the portions of the battery electrode material; and implementing a second convolutional neural network and performing deep learning to classify the defects.

In other features, the defect detection method further includes, when classifying the defects, determining whether each of the defects is a dark band defect, a contamination defect, a crack, an embrittlement defect, a wrinkle, an edge imperfection, a polka dot, a void, or a delamination.

In other features, the defect detection method further includes analyzing portions of the at least one of the feature maps and image data associated with the portions of the battery electrode material that include defects and refraining from analyzing portions of the at least one of the feature maps and image data associated with the portions of the battery electrode material that do not include defects.

In other features, the defect detection method further includes: prior to the coarse detector determining whether there are defects in portions of the battery electrode material, performing a processing procedure on the image data received from the sensors, where the processing procedure comprises at least one of aligning the image data, normalizing the image data, synchronizing the image data, and cropping the image data to generate processed data; and generating fused data of the feature maps based on the processed data.

In other features, the processing procedure includes aligning the image data, normalizing the image data, synchronizing the image data, and cropping the image data.

In other features, the classifying of the defects includes performing i) multi-classification to identify defect types, and ii) localization to locate and scale bounding boxes to defects present in an image.

In other features, the classifying of the defects includes analyzing portions of the at least one of the feature maps and image data containing at least one defect and refraining from analyzing portions of the at least one of the feature maps and image data not containing a defect.

Further areas of applicability of the present disclosure will become apparent from the detailed description, the claims and the drawings. The detailed description and specific examples are intended for purposes of illustration only and are not intended to limit the scope of the disclosure.

### BRIEF DESCRIPTION OF THE DRAWINGS

The present disclosure will become more fully understood from the detailed description and the accompanying drawings, wherein:

FIG. 1 is a perspective view of a manufacturing system performing a roll-to-roll electrode coating and drying process and electrode film material scanning with multiple sensors in accordance with the present disclosure;

FIG. 2 is a perspective view of a manufacturing system performing roll-to-roll electrode calendering processing and electrode film material scanning with multiple sensors in accordance with the present disclosure;

FIG. 3 is a functional block diagram of a multi-sensor-based battery electrode scanning system illustrating detection of different types of defects and corresponding classification in accordance with the present disclosure;

FIG. 4 is a perspective view of an x-ray scanner scanning battery electrode material in accordance with the present disclosure;

FIG. 5 is a side cross-sectional view of a terahertz scanner scanning battery electrode material in accordance with the present disclosure;

FIG. 6 is a functional block diagram of a multi-sensor-based battery electrode scanning system illustrating data fusion and feature extraction for corresponding grid sections of images from multiple sensors and corresponding classification in accordance with the present disclosure; and

FIG. 7 illustrates a battery electrode material scanning method in accordance with the present disclosure.

In the drawings, reference numbers may be reused to identify similar and/or identical elements.

### DETAILED DESCRIPTION

Electric power sources, such as LV and HV batteries and battery packs for vehicles, include electrodes, referred to as cathodes and anodes. As an example, a nickel manganese cobalt (NMC) cathode of lithium-ion batteries used in electric vehicles can be made of materials such as lithium, nickel, manganese, and cobalt oxides. A NMC cathode can be formed by depositing a mixture of the aforementioned metal oxides evenly onto a layer of aluminum foil to form a layered stack and compressing (or calendering) the stack between a pair of rollers. The pressure applied by the rollers to compress the stack can be adjusted.

During production of battery electrodes, defects can occur and/or exist in the electrode material, such as surface, internal and interface type defects. For example, many types of defects can occur in roll-to-roll coating, drying and calendering processes. The defects are formed by different mechanisms and differ in appearance, location and size. The stated defects can include polka dot, dark band, delamination, contamination, embrittlement, broken edge, and wrinkle defects. The defects can also include cracks in electrode material. Polka dot defects refer to circular and/or irregular shaped spots on the material, which are discolored and typically exist in clusters. Dark band defects refer to bands (or strips) in the material that are darker in color. Embrittlement defects refer to warped, irregular and/or uneven portions of the material. Embrittlement defects typically occur near edges of the material. Delamination refers to separation in layers of a material stack. The electrode material is a stack of layers. A broken edge defect refers to an edge having portions that are deformed or missing. A wrinkle defect refers to a defect that has a regular and/or repeating pattern (e.g., a wave). A wrinkle may have an associated period. The defects can negatively affect capacity of formed electrodes.

The examples set forth herein include a battery electrode scanning system that scans battery electrode material using multiple sensors of different types for surface, interior, and interface defect detection. The system performs in-process automatic detection of all types and levels of defects. The system fuses data from the sensors and performs feature extraction based on the fused data to detect and classify defects of various different types anywhere in the battery electrode material.

The sensing technologies of the sensors incorporated in the disclosed system are primarily applicable for either surface, interior, or interface defect detection. Multiple sensors (three or more sensors) are used, where at least one sensor is primarily applicable for each of surface defect detection, interior defect detection, and interface defect detection. Multi-sensor data fusion as disclosed herein leverages the strengths of the different sensing technologies for improved scanning, detecting, classifying, and reporting of defects. Deep learning is performed to solve complex pattern recognition problems to detect electrode defects based on large amounts of detected data.

Although the examples described herein are primarily directed to detecting defects within battery electrode material, the examples may be applied to other materials. The examples may be applied to electrode material and/or to electrodes. The examples may be applied to high-resolution and high-speed roll-to-roll coating, drying, and calendering processes. In an embodiment, sensors are selected and used to monitor and detect surface, interior (or bulk stack), and interface defects of electrode material. A bulk stack may refer to one or more stacked layers, which may be coated. Sensor data is collected from the sensors and fused and processed using deep learning methods to create multi-scale feature maps that include different levels of graphical feature abstraction of the original sensor data. Subsequently, a two-step coarse-to-fine deep learning procedure is implemented to parse the feature maps to achieve maximum efficiency and accuracy and enable high-speed and real-time defect detection.

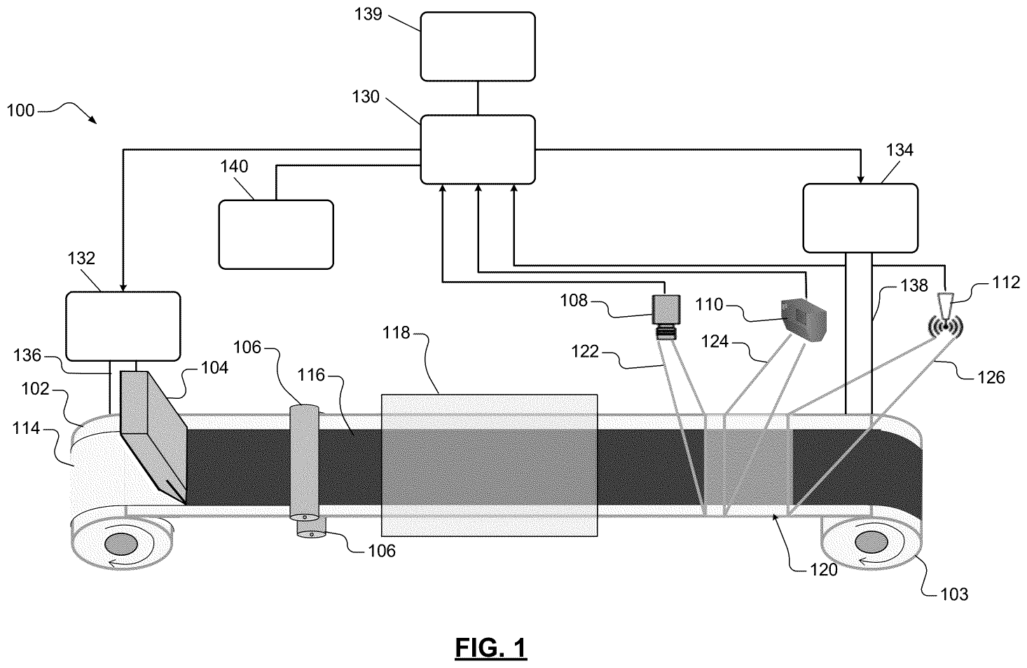

FIG. 1 shows a perspective view of a manufacturing system 100 performing a roll-to-roll electrode coating and drying process and electrode film material scanning with multiple sensors. The manufacturing system 100 includes material unwinding and winding rollers 102, 103, a slot die (or coating applicator) 104, guide rollers 106, and sensors (or sensor systems) 108, 110, 112. Although three sensors are shown, more sensors may be included. Material (e.g., battery electrode material) 114 is moved from the roller 102 to the roller 103 through the guide rollers 106. The slot die 104 applies a coating 116 on the material 114. The material 114 may be electrode foil material, which is coated and passed through a drying zone 118. Subsequent to drying, the coated material is then scanned by the sensors 108, 110, 112. A scanning area 120 is shown representing an area between the field-of-view (FOV) of the first sensor 108 and a FOV of a last sensor (or sensor 112). Distances between the FOVs of the sensors 108, 110, 112 may vary.

The sensors 108, 110, 112 may be of various types. In an embodiment, the sensor 108 is a surface scanning sensor, the sensor 110 is an interior scanning sensor, and the sensor 112 is an interface scanning sensor. The order of the sensors may be different than shown. The sensors 108, 110, 112 operate in different inspection modes and are used for comprehensive defect detection. FOVs of the sensors 108, 110, 112 are designated 122, 124, 126, respectively.

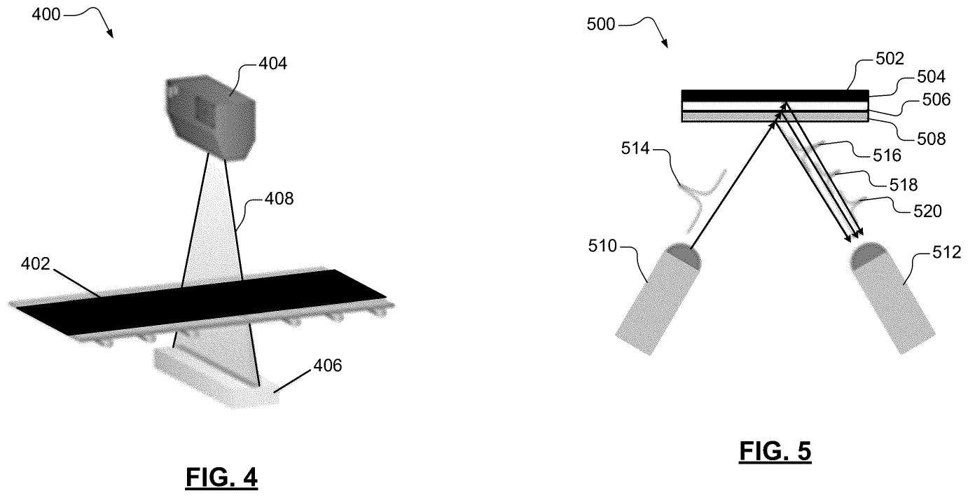

The sensor 108 operates in a surface profile inspection mode to detect surface characteristics and quality including detecting defects such as dark band defects, contamination defects, cracks, embrittlement defects, wrinkles, edge imperfections, etc. The more defects per area, the larger the defects, etc., the poorer the quality of the material. The sensor 108 may be implemented as a line scanning camera, a laser two-dimensional (2D) profiler, a laser three-dimensional (3D) profiler, a flash thermography scanner, etc. The 2D and 3D laser profilers may be used to monitor electrode profile defects and geometrical characteristics. The sensor 110 operates in an internal inspection mode to detect internal characteristics and defects including polka dot defects, cracks, voids, etc. The sensor 110 may be implemented as an X-ray scanner, a neutron imaging sensor, an eddy current sensor, a beta gauging sensor, etc. An example of an X-ray scanner is shown in FIG. 4. The sensor 112 operates in an interfacial inspection mode to detect interface characteristics and defects such as cracks, delamination defects, etc. The sensor 112 may be implemented as a terahertz scanner, an ultrasound sensor, or other sensor capable of operating in an interfacial inspection mode. An example of a terahertz scanner is shown in FIG. 5.

The manufacturing system 100 further includes a control module 130, actuators and motors 132, and actuators and motors 134. The actuators and motors 132 may rotate and/or move the roller 102 via a shaft 136. The actuators and motors 134 may rotate and/or move the roller 103 via a shaft 138. The control module 130 receives outputs of the sensors 108, 110, 112 and performs the data fusing and classification methods described below. The control module 130 may control the actuators and motors 132, 134 including activation, deactivation, speed, and/or movement of the rollers 102, 103.

In order to know where defects are detected, positions and speeds of the shafts 136, 138 may be monitored. Position encoders may be used to track the positions of the shafts 136, 138 and as a result position of the battery electrode material relative to the sensors 108, 110, 112 as the battery electrode material is moved between the rollers 102, 103. Defects and corresponding information may be displayed on display 139. In an embodiment, the manufacturing system 100 includes a cutter 140 (or cutting system) that may be controlled by the control module 130 to cut off defective portions of the coated battery electrode material. This may occur between the rollers 102, 103 or elsewhere. The coated battery electrode material may be fed to the cutter 140 and the cutter 140 may then remove the material having defects.

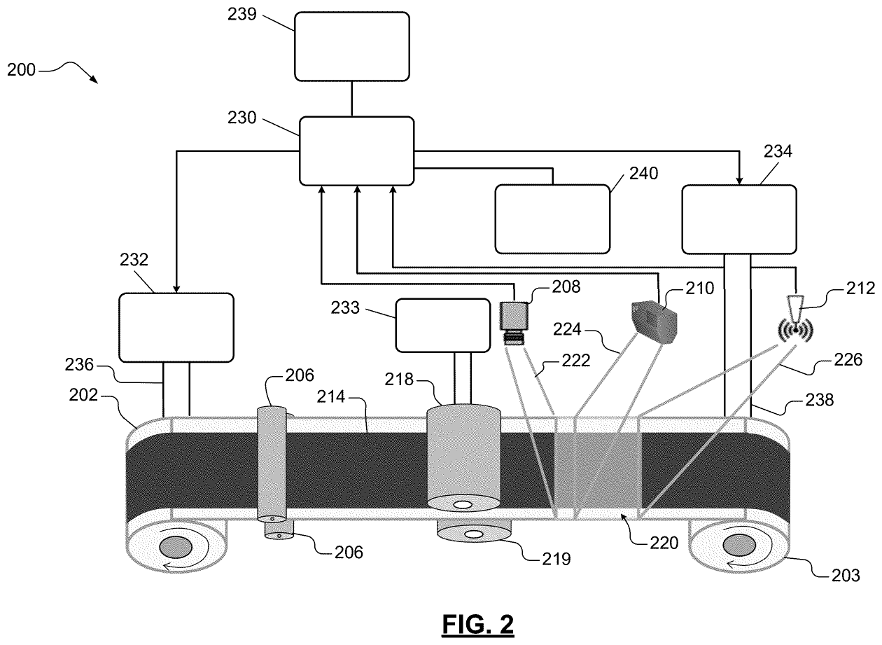

FIG. 2 shows a perspective view of a manufacturing system 200 performing roll-to-roll electrode calendering processing and electrode film material scanning with multiple sensors. The manufacturing system 200 may be configured similarly as the system 100 of FIG. 1 and includes material moving rollers 202, 203, guide rollers 206, and sensors (or sensor systems) 208, 210, 212, which may be configured and operate similarly as the sensors 108, 110, 112 of FIG. 1. Although three sensors are shown, more sensors may be included.

Material (e.g., battery electrode material) 214 is moved from the roller 202 to the roller 203 through the guide rollers 206. The material 214 is passed between calendering rollers 218, 219 and is then scanned by the sensors 208, 210, 212. The calendering rollers 218, 219 apply pressure on the material 214. A scanning area 220 is shown representing an area between the FOV of the first sensor 208 and a FOV of a last sensor (or sensor 212). Distances between the FOVs of the sensors 208, 210, 212 may vary.

The sensors 208, 210, 212 may be of various types. In an embodiment, the sensor 208 is a surface scanning sensor, the sensor 210 is an interior scanning sensor, and the sensor 212 is an interface scanning sensor. The order of the sensors may be different than shown. The sensors 208, 210, 212 operate in different inspection modes and are used for comprehensive defect detection. FOVs of the sensors 208, 210, 212 are designated 222, 224, 226, respectively.

The sensor 208 operates in a surface profile inspection mode to detect surface defects including dark band defects, contamination defects, cracks, embrittlement defects, wrinkles, edge imperfections, etc. The sensor 208 may be implemented as a line scanning camera, a laser 3D profiler, a flash thermography scanner, etc. The sensor 210 operates in an internal inspection mode to detect internal defects including cracks, polka dot defects, cracks, voids, etc. The sensor 210 may be implemented as an X-ray scanner, a neutron imaging sensor, an eddy current sensor, a beta gauging sensor, etc. An example of an X-ray scanner is shown in FIG. 4. The sensor 212 operates in an interfacial inspection mode to detect cracks, delamination defects, etc. The sensor 212 may be implemented as a terahertz scanner, an ultrasound sensor, or other sensors capable of operating in an interfacial inspection mode. An example of a terahertz scanner is shown in FIG. 5.

The manufacturing system 200 further includes a control module 230, actuators and motors 232, actuators and motors 233, and actuators and motors 234. The actuators and motors 232 may rotate and/or move the roller 202 via a shaft 236. The actuators and motors 233 may control distance between the rollers 218, 220 and pressure applied by the rollers 218, 220 on the battery electrode material 214. The actuators and motors 234 may rotate and/or move the roller 203 via a shaft 238. The control module 230 receives outputs of the sensors 208, 210, 212 and performs the data fusing and classification methods described below. The control module 230 may control the actuators and motors 232, 233, 234 including activation, deactivation, speed, applied pressure, and/or movement of the rollers 202, 203, 218, 220.

Defects and corresponding information may be displayed on display 239. In an embodiment, the manufacturing system 200 includes a cutter 240 (or cutting system) that may be controlled by the control module 230 to cut off defective portions of the coated battery electrode material. This may occur between the rollers 202, 203 or elsewhere. The coated battery electrode material may be fed to the cutter 240 and the cutter 240 may then remove the material having defects.

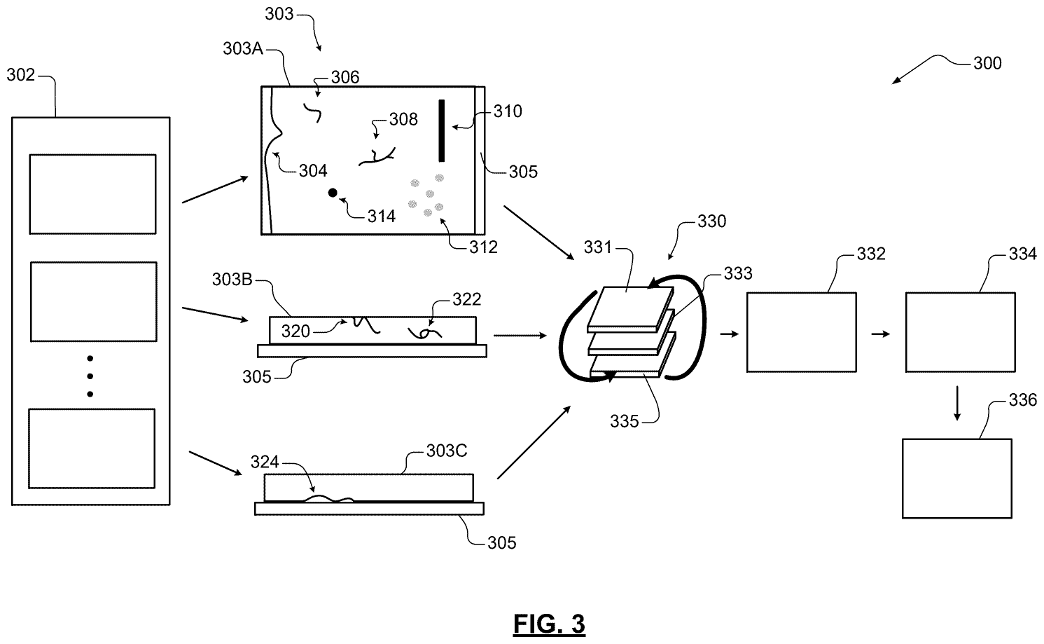

FIG. 3 shows a multi-sensor-based battery electrode scanning system 300 illustrating detection of different types of defects and corresponding classification. The multi-sensor-based battery electrode scanning system 300 includes sensors 302, such as the sensors of FIGS. 1 and 2. At least three different types of sensors are included. The sensors 302 perform in-line measuring and detect defects in battery electrode material 303 being scanned. A top view 303A and side cross-sections views 303B, 303C of the battery electrode material 303 are shown. The battery electrode material may be disposed on a conveyor belt 305. One or more of the sensors 302 may detect surface defects such as an edge imperfection 304, a contamination 306, a crack 308, a dark band 310, an agglomeration 312, polka dot defects 314, or other surface defects. One or more other ones of the sensors 302 may detect interior defects such as a crack 320, a void 322, or other interior defects. One or more other ones of the sensors 302 may detect interface defects such as delamination defect 324.

Data generated by the sensors 302 is fused together during sensor fusion operations, designated 330 and further described below. Surface data 331, interior data 333 and interface data 335 are combined including surface defect data, interior defect data, and interface defect data. The interior data 333 may be referred to as bulk material data. Subsequent to sensor fusion, a two-step defect detection procedure is implemented including performing coarse defect detection and classification 332 and fine defect detection and classification 334. The coarse defect detection and classification 332 includes indicating whether a certain portion of the battery electrode material includes a defect or does not include a defect and thus deemed normal. The coarse defect detection and classification 332 may indicate a High (or 1) or Low (or 0) value depending on whether a certain portion of the battery electrode material has a defect. The fine defect detection and classification 334 includes a more detailed determination of the type of defect detected and location of the defect. In addition to the type and location, this may include identifying aspects about the defect, such as size shape, pattern, depth, etc. A defect map 336 may be generated based on results of the fine defect detection and classification 334. The defect map may include some or all of the information determined from the fine defect detection and classification 334. In an embodiment, the defect map is an image showing the defects in respective locations of the battery electrode material.

FIG. 4 shows a perspective view of an X-ray scanner 400 scanning battery electrode material 402. The X-ray scanner 400 includes an X-ray generator (or source) 404 and an X-ray detector 406. The X-ray generator has a FOV (or X-ray beam) 408 that is received at the X-ray detector 406 after passing through the battery electrode material 402.

FIG. 5 shows a side cross-sectional view of a terahertz scanner 500 scanning battery electrode material 502 including layers 504, 506, 508. The terahertz scanner 500 is an electromagnetic imaging system. The terahertz scanner 500 includes an emitter 510 and a detector 512. The emitter 510 generates a terahertz pulse 514 that is directed at the battery electrode material 502 and is reflected at the point of incidence, at an interface between the layers 504 and 506, and at an interface between layers 506 and 508. The reflected pulses are designated 516, 518, 520.

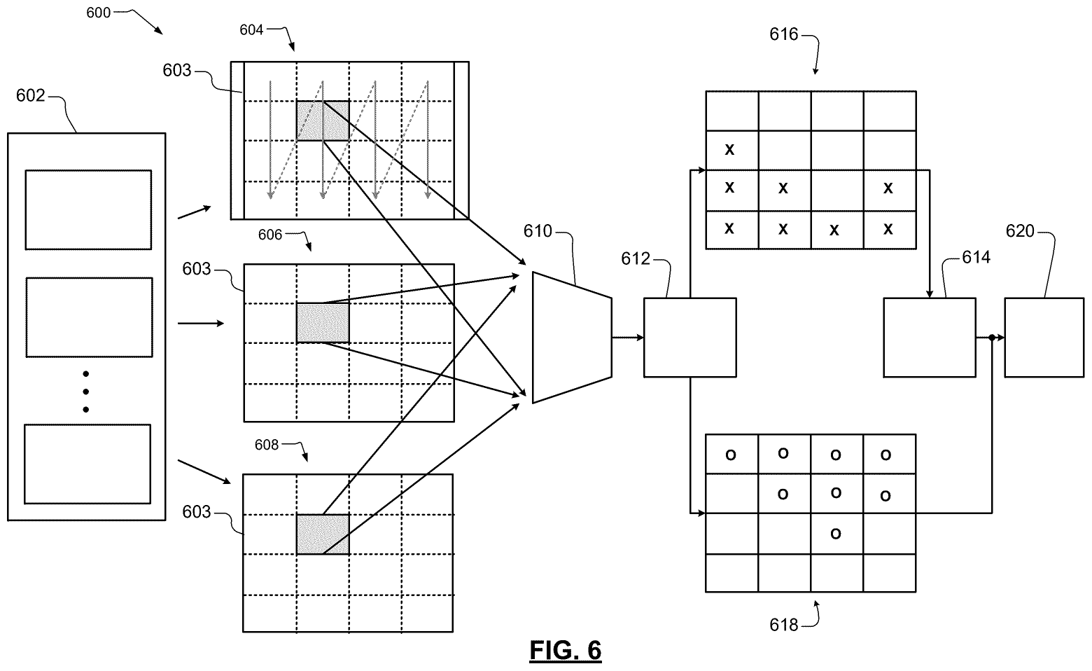

FIG. 6 shows a multi-sensor-based battery electrode scanning system 600 illustrating data fusion and feature extraction for corresponding grid sections of images from multiple sensors and corresponding classification. The system 600 includes sensors 602, such as the sensors of FIGS. 1 and 2. At least three different types of sensors are included. The sensors 602 perform in-line measuring and detect defects in battery electrode material 603 being scanned.

The sensors 602 provide at least three different types of images respectively for the three different sensor technologies of the at least three different types of sensors. Three example images 604, 606, 608 are shown and may be respectively an optical image from a surface scanning sensor, a radiography image from an interior scanning sensor, and a terahertz image from an interface scanning and/or terahertz sensor. The radiography image may be an X-ray image. During sensor fusion, the images from the sensors are provided to a feature extractor module 610.

The feature extractor module 610 may implement a convolutional neural network and perform deep learning to extract features from the images corresponding to defects and construct multi-scale feature maps of monitored data for defect detection. The feature extraction is performed by deep learning networks (e.g., convolution neural networks) on multi-sensor data to generate a fused feature map. The feature extraction networks use different kernel sizes to capture defects at different scales. The fused feature map has sufficient channels, to keep feature rich information extracted from sensory data. The features may refer to features of portions of the battery electrode material with irregularities and features of portions of the battery electrode material without irregularities. The features may refer to colors, patterns, shapes, brightness, locations, etc. and/or other parameters and/or associated values that are indicative of aspects of the battery electrode material and quality thereof.

In the example shown, a coarse detector 612 performs coarse detection to determine whether each segmented portion of the battery electrode material is a defect. An example defect grid 616 and an example non-defect grid 618 for a portion of the battery electrode material are shown. The defect grid 616 indicates which sections of the grid have defects. The non-defect grid 618 indicates which sections of the grid do not include defects. The feature extractor module 610, the coarse detector 612 and the fine detector 614 may be implemented by any of the control modules referred to herein. The fine detector 614 may review one or more of the grids 616, 618 and, based on the feature maps (or image representations) out of the feature extractor module 610, generates a defect map 620.

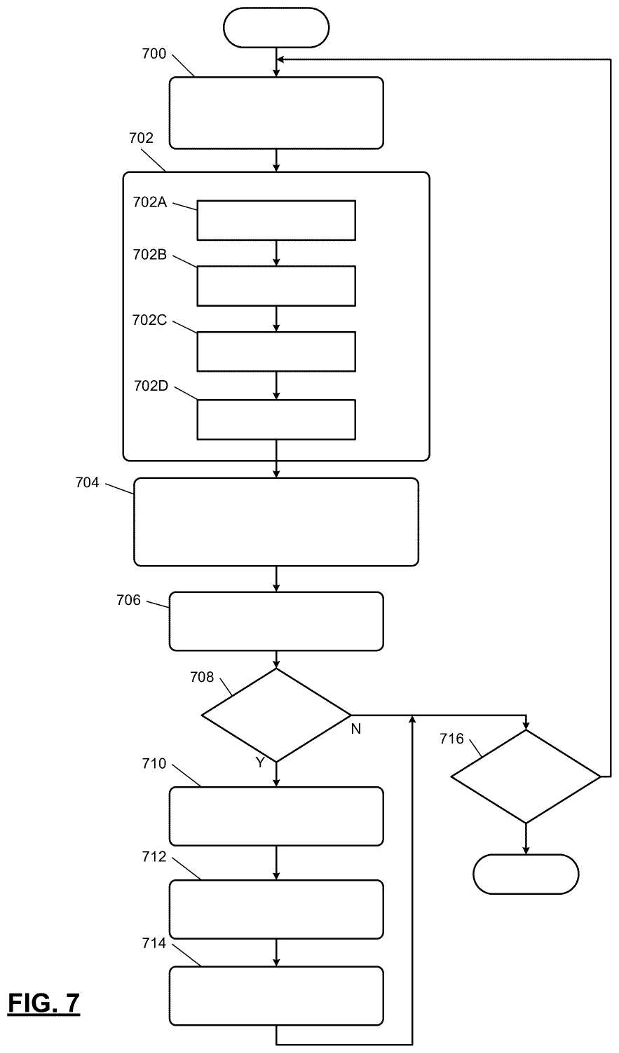

FIG. 7 shows a battery electrode material scanning method. The following operations may be performed by a control module, such as one of the control modules of FIGS. 1-2 and may implement the processes described with respect to FIGS. 3 and 6.

At 700, the control module performs in-line measuring including collecting sensor data from multiple sensors, such as from any of the sensors referred to herein including sensor data from at least a surface scanning sensor, an interior scanning sensor, and an interface scanning sensor.

At 702, the control module performs a data processing procedure. At 702A, the control module performs an alignment including transforming surface, interior, and interface measurements (or data) into a unified reference frame. This includes spatially aligning data from the multiple sensors, as each of the sensors may have a different size FOV and/or a viewing angle and thus data from one or more sensors may be scaled or shifted to be aligned with data from another one or more sensors. Also, the sensors may have respective coordinate systems. The control module may perform a data transformation on data from one or more sensors to be in a same coordinate system as data from another one or more sensors. The feature extractor module 610 may be implemented and use different kernel sizes to capture defects at different scales. The kernel sizes refer to different scales of feature maps. The kernel sizes may also refer to filter sizes in convolution neural networks and/or deep learning networks. As an example, if a defect is small, then the data associated with the defect may be extracted in a small-scale feature map. Similarly, if a defect is large, then the data associated with the defect may be extracted in a large-scale feature map.

At 702B, the control module may perform normalization including normalizing the data and/or units of measure of the data for the measurements taken. This may include normalizing the data between 0 and 1 and/or converting the data to have the same units of measure.

At 702C, the control module synchronizes the data from the sensors. The data collected from each sensor for a particular location on battery electrode material may have been captured at different times. The control module offsets (or delays) data from one or more sensors in time to be synchronized with data from another one or more sensors. This may include shifting (or offsetting), advancing, and/or delaying in time data from one or more sensors to be aligned with data from one or more other sensors. All measurements made with respect to an electrode region are made consistent in time.

At 702D, the control module crops collected data into smaller data patches to alleviate computational complexity of sensor fusion and feature detection. The collected data may be grouped into small data sets associated with respective portions of the battery electrode material, such as portions associated with grid boxes. For example, if the battery electrode material is divided by grid lines into row and column sections, each of the small data sets may be associated with one of the row and column boxes of the grid.

At 704, the control module performs sensor fusion operations to fuse sensor data from the multiple sensors and perform feature extraction to provide one or more fused multi-scaled feature maps (or one or more feature representation images), as described above. The feature maps have areas in which features associated with defects are located. Different features (or aspects) of an image, referred to as channels, are combined to create a resultant fused feature map. The control module may perform a deep learning algorithm to fuse and encode sensor data when generating the multi-scaled feature maps. Each of the maps is a scaled version of a feature extraction and may have a corresponding small, medium or large-scale size.

The following two step (coarse and fine) detection and classification approach of operations 706 and 710 reduce computational complexity for deep network training and for making inferences, and thus improves efficiency of defect detection. The two-step approach also improves accuracy of defect detection because the data used for fine feature detection are the data samples including defects, not the data samples having no defects. The data samples refer to the feature maps (or image representations).

At 706, the control module performs coarse defect detection and classification as described herein and generates binary information. Coarse detection is enabled by binary deep learning classification, which distinguishes defective samples from normal samples at a high speed for improved efficiency. The binary information indicating whether each portion (e.g., grid section) of the battery electrode material is defective or normal. The control module may perform deep learning and compare current feature maps to stored sample sets associated with particular defects to determine whether defects exist in each of the associated portions of the battery electrode material scanned. The control module implements a first convolutional neural network (CNN) that may rate abstract image representation received from the feature extractor and classify the image representation as “defective” or “normal”. The first CNN may output a probability percentage indicating whether the image representation belongs to the defective classification or the normal classification. The control module may indicate which grid sections of the battery electrode material (or scanned area) include defects and which sections do not include defects.

At 708, the control module determines whether one or more defects have been detected. If yes, operation 710 is performed, otherwise operation 716 is performed.

At 710, the control module performs fine defect detection and classification as described herein based on the binary information and at least one of the feature maps and image data. The control module may implement a second convolution neural network to determine whether the image representation belongs to a certain class and/or type of defects. The classes of defect may refer to whether a defect is a surface defect, an interior defect, or an interface defect. The types of defects may refer to whether a defect is a dark band defect, a contamination defect, a crack, an embrittlement defect, a wrinkle, an edge imperfection, a polka dot defect, a void, a delamination defect, etc. Fine defect detection includes performing two operations concurrently and possibly simultaneously. The two operations are multi-classification and localization. Multi-classification is performed to identify defect types (polka dot, dark band, crack, contamination, delamination, etc.). Localization is performed to locate and scale bounding boxes to defects present in an image. The scaled boxes may be analyzed when classifying the defects, which are associated with certain portions of the battery electrode material, not other portions of the battery electrode material. In an embodiment, the two-step approach improves defect detection accuracy because the data used for the fine feature detection is the data samples that include defects, not the data samples that do not include defects. The scaled boxes include the data samples with defects.

At 712, the control module outputs a defect classification and location identification map. This map may be reported to a remote network device and/or displayed on a display (e.g., one of the displays 139, 239 of FIGS. 1-2).

At 714, the control module may perform a defect discarding procedure and/or a future defect avoidance procedure. For example, he control module may discard one or more defects. The control module may determine the quality of each portion (or grid section) of battery electrode material. Quality values may be generated based on the defect detected, confidence values associated with the defects, the number of defects per area, the size of the defects, the types of the defects, the locations of the defects, etc. The control module may then determine whether certain portions ought to be discarded based on the quality values. In a simplified embodiment, if a portion has one or more defects, the portion is discarded. In another more complex embodiment, quality values are generated as stated and the control module determines which portions to discard based on the quality values.

The control module may report and/or display which one or more defects should be cut off from the battery electrode material and discarded. The report may be sent to a network device remotely located from the control module. In an embodiment, the control module waits for approval prior to proceeding with removing defective material. The approval may come via the display or other input device or from the network device that received the report.

In an embodiment, the control module controls movement of the battery electrode material to a location of the defect and indicates where the defect is located, such that a portion of the battery electrode material having the defect can be cut off. This may include reversing the direction of rollers (e.g., the rollers 102, 103, 202, 203 of FIGS. 1-2). The control module may control a cutter to cut the material and/or a marker to mark the area (or portion) to be removed. In an embodiment, when a defect is removed, one or more rows of material are cutoff and discarded.

As another example, feedback control may be implemented such that the control module performs certain operations based on the detected and classified defects to prevent occurrence of similar defects in the future. For example, the control module may, based on the sensor data and defect classifications and apart from discarding the battery electrode material with defects by using a cutter, the control module performs feedback control. This may include performing process control adjustments and/or roller adjustments to avoid occurrence of at least some of the defects from appearing in subsequent battery electrode material. The control module may, for example, adjust pressure applied by the calendering rollers, speed of the winding and unwinding rollers, etc. This may be applicable to certain types of defects.

At 716, the control module determines whether there is more sample area of the battery electrode material to scan. If yes, operation 700 may be performed, otherwise the method may end.

The above-described examples provide timely in-process real-time defect detection for quality assurance in large scale electrode production. In-process monitoring systems are disclosed for battery electrode manufacturing that can detect surface, interior (or bulk material), and interface electrode defects concurrently. Multiple sensors of different types are integrated to monitor the quality of electrode surface interior (or bulk material), and interface concurrently. Deep learning-based sensor fusion methods are used to construct multi-scale feature representations and to create comprehensive quality data for defect detection. Defect detection is performed by performing a coarse-to-fine deep learning method for efficient and accurate results.

The foregoing description is merely illustrative in nature and is in no way intended to limit the disclosure, its application, or uses. The broad teachings of the disclosure can be implemented in a variety of forms. Therefore, while this disclosure includes particular examples, the true scope of the disclosure should not be so limited since other modifications will become apparent upon a study of the drawings, the specification, and the following claims. It should be understood that one or more steps within a method may be executed in different order (or concurrently) without altering the principles of the present disclosure. Further, although each of the embodiments is described above as having certain features, any one or more of those features described with respect to any embodiment of the disclosure can be implemented in and/or combined with features of any of the other embodiments, even if that combination is not explicitly described. In other words, the described embodiments are not mutually exclusive, and permutations of one or more embodiments with one another remain within the scope of this disclosure.

Spatial and functional relationships between elements (for example, between modules, circuit elements, semiconductor layers, etc.) are described using various terms, including “connected,” “engaged,” “coupled,” “adjacent,” “next to,” “on top of,” “above,” “below,” and “disposed.” Unless explicitly described as being “direct,” when a relationship between first and second elements is described in the above disclosure, that relationship can be a direct relationship where no other intervening elements are present between the first and second elements, but can also be an indirect relationship where one or more intervening elements are present (either spatially or functionally) between the first and second elements. As used herein, the phrase at least one of A, B, and C should be construed to mean a logical (A OR B OR C), using a non-exclusive logical OR, and should not be construed to mean “at least one of A, at least one of B, and at least one of C.”

In the figures, the direction of an arrow, as indicated by the arrowhead, generally demonstrates the flow of information (such as data or instructions) that is of interest to the illustration. For example, when element A and element B exchange a variety of information but information transmitted from element A to element B is relevant to the illustration, the arrow may point from element A to element B. This unidirectional arrow does not imply that no other information is transmitted from element B to element A. Further, for information sent from element A to element B, element B may send requests for, or receipt acknowledgements of, the information to element A.

In this application, including the definitions below, the term “module” or the term “controller” may be replaced with the term “circuit.” The term “module” may refer to, be part of, or include: an Application Specific Integrated Circuit (ASIC); a digital, analog, or mixed analog/digital discrete circuit; a digital, analog, or mixed analog/digital integrated circuit; a combinational logic circuit; a field programmable gate array (FPGA); a processor circuit (shared, dedicated, or group) that executes code; a memory circuit (shared, dedicated, or group) that stores code executed by the processor circuit; other suitable hardware components that provide the described functionality; or a combination of some or all of the above, such as in a system-on-chip.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

The term code, as used above, may include software, firmware, and/or microcode, and may refer to programs, routines, functions, classes, data structures, and/or objects. The term shared processor circuit encompasses a single processor circuit that executes some or all code from multiple modules. The term group processor circuit encompasses a processor circuit that, in combination with additional processor circuits, executes some or all code from one or more modules. References to multiple processor circuits encompass multiple processor circuits on discrete dies, multiple processor circuits on a single die, multiple cores of a single processor circuit, multiple threads of a single processor circuit, or a combination of the above. The term shared memory circuit encompasses a single memory circuit that stores some or all code from multiple modules. The term group memory circuit encompasses a memory circuit that, in combination with additional memories, stores some or all code from one or more modules.

The term memory circuit is a subset of the term computer-readable medium. The term computer-readable medium, as used herein, does not encompass transitory electrical or electromagnetic signals propagating through a medium (such as on a carrier wave); the term computer-readable medium may therefore be considered tangible and non-transitory. Non-limiting examples of a non-transitory, tangible computer-readable medium are nonvolatile memory circuits (such as a flash memory circuit, an erasable programmable read-only memory circuit, or a mask read-only memory circuit), volatile memory circuits (such as a static random access memory circuit or a dynamic random access memory circuit), magnetic storage media (such as an analog or digital magnetic tape or a hard disk drive), and optical storage media (such as a CD, a DVD, or a Blu-ray Disc).

The apparatuses and methods described in this application may be partially or fully implemented by a special purpose computer created by configuring a general purpose computer to execute one or more particular functions embodied in computer programs. The functional blocks, flowchart components, and other elements described above serve as software specifications, which can be translated into the computer programs by the routine work of a skilled technician or programmer.

The computer programs include processor-executable instructions that are stored on at least one non-transitory, tangible computer-readable medium. The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc.

The computer programs may include: (i) descriptive text to be parsed, such as HTML (hypertext markup language), XML (extensible markup language), or JSON (JavaScript Object Notation) (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C #, Objective-C, Swift, Haskell, Go, SQL, R, Lisp, Java®, Fortran, Perl, Pascal, Curl, OCaml, Javascript®, HTML5 (Hypertext Markup Language 5th revision), Ada, ASP (Active Server Pages), PHP (PHP: Hypertext Preprocessor), Scala, Eiffel, Smalltalk, Erlang, Ruby, Flash®, Visual Basic®, Lua, MATLAB, SIMULINK, and Python®.

What is claimed is:

1. A defect detection system comprising:
a plurality of sensors of different types configured to scan battery electrode material and generate a plurality of output signals including respective image data;
a feature extractor module configured to receive the plurality of output signals and perform feature extraction to fuse the image data of the plurality of output signals to generate feature maps for respective portions of the battery electrode material;
a coarse detector configured, based on the feature maps, to determine whether there are defects in the portions of the battery electrode material, and generate binary information for each of the portions indicating whether the portions include one or more defects;
a fine detector configured, based on the binary information and at least one of the feature maps and image data, to classify the defects; and
a control module configured to at least one of i) report the classified defects, ii) determine whether one or more of the portions of the battery electrode material which have one or more defects ought to be discarded, iii) perform operations to discard the one or more portions of the battery electrode material which have one or more defects, and iv) perform feedback process control operations to prevent future occurrences defect similar to the classified defects.

2. The defect detection system of claim 1, wherein the plurality of sensors comprise at least one surface scanning sensor, at least one interior scanning sensor, and at least one interface scanning sensor.

3. The defect detection system of claim 2, wherein:
the at least one surface scanning sensor comprises at least one of a camera, a laser 3D profiler, and a flash thermography scanner;
the at least one interior scanning sensor comprises at least one of an X-ray scanner, a neutron imaging sensor, an eddy current sensor, and a beta gauging sensor; and
the at least one interface scanning sensor comprises at least one of a terahertz scanner and an ultrasound sensor.

4. The defect detection system of claim 1, wherein:
the coarse detector is configured to implement a first convolutional neural network and perform deep learning to determine whether there are defects in the portions of the battery electrode material; and
the fine detector is configured to implement a second convolutional neural network and perform deep learning to classify the defects.

5. The defect detection system of claim 1, wherein the fine detector is configured, when classifying the defects, to determine whether each of the defects is a dark band defect, a contamination defect, a crack, an embrittlement defect, a wrinkle, an edge imperfection, a polka dot, a void, or a delamination.

6. The defect detection system of claim 1, wherein the fine detector analyzes portions of the at least one of the feature maps and image data associated with the portions of the battery electrode material that include defects and does not analyze portions of the at least one of the feature maps and image data associated with the portions of the battery electrode material that do not include defects.

7. The defect detection system of claim 1, wherein:
the control module is configured, prior to the coarse detector determining whether there are defects in portions of the battery electrode material, to perform a processing procedure on the image data received from the plurality of sensors;
the processing procedure comprises at least one of aligning the image data, normalizing the image data, synchronizing the image data, and cropping the image data to generate processed data; and
the control module is configured to generate fused data of the feature maps based on the processed data.

8. The defect detection system of claim 7, wherein the processing procedure includes aligning the image data, normalizing the image data, synchronizing the image data, and cropping the image data.

9. The defect detection system of claim 1, wherein the fine detector is configured to perform i) multi-classification to identify defect types, and ii) localization to locate and scale bounding boxes to defects present in an image.

10. The defect detection system of claim 1, wherein the fine detector is configured to analyze portions of the at least one of the feature maps and image data containing at least one defect and to not analyze portions of the at least one of the feature maps and image data not containing a defect.

11. A defect detection method comprising:
scanning battery electrode material, via a plurality of sensors of different types, and generating a plurality of output signals including respective image data;
receiving the plurality of output signals and performing feature extraction to fuse the image data of the plurality of output signals to generate feature maps for respective portions of the battery electrode material;
based on the feature maps, determining whether there are defects in the portions of the battery electrode material, and generate binary information for each of the portions indicating whether the portions include one or more defects;
based on the binary information and at least one of the feature maps and image data, classifying the defects; and
at least one of i) reporting the classified defects, ii) determining whether one or more of the portions of the battery electrode material which have one or more defects ought to be discarded, iii) performing operations to discard the one or more portions of the battery electrode material which have one or more defects, and iv) performing feedback process control operations to prevent future occurrences defect similar to the classified defects.

12. The defect detection method of claim 11, wherein the plurality of sensors comprise at least one surface scanning sensor, at least one interior scanning sensor, and at least one interface scanning sensor.

13. The defect detection method of claim 12, wherein:
the at least one surface scanning sensor comprises at least one of a camera, a laser 3D profiler, and a flash thermography scanner;
the at least one interior scanning sensor comprises at least one of an X-ray scanner, a neutron imaging sensor, an eddy current sensor, and a beta gauging sensor; and
the at least one interface scanning sensor comprises at least one of a terahertz scanner and an ultrasound sensor.

14. The defect detection method of claim 11, further comprising:
implementing a first convolutional neural network and performing deep learning to determine whether there are defects in the portions of the battery electrode material; and
implementing a second convolutional neural network and performing deep learning to classify the defects.

15. The defect detection method of claim 11, further comprising, when classifying the defects, determining whether each of the defects is a dark band defect, a contamination defect, a crack, an embrittlement defect, a wrinkle, an edge imperfection, a polka dot, a void, or a delamination.

16. The defect detection method of claim 11, further comprising analyzing portions of the at least one of the feature maps and image data associated with the portions of the battery electrode material that include defects and refraining from analyzing portions of the at least one of the feature maps and image data associated with the portions of the battery electrode material that do not include defects.

17. The defect detection method of claim 11, further comprising:
prior to the coarse detector determining whether there are defects in portions of the battery electrode material, performing a processing procedure on the image data received from the plurality of sensors,
wherein the processing procedure comprises at least one of aligning the image data, normalizing the image data, synchronizing the image data, and cropping the image data to generate processed data; and
generating fused data of the feature maps based on the processed data.

18. The defect detection method of claim 17, wherein the processing procedure includes aligning the image data, normalizing the image data, synchronizing the image data, and cropping the image data.

19. The defect detection method of claim 11, wherein classifying the defects comprises performing i) multi-classification to identify defect types, and ii) localization to locate and scale bounding boxes to defects present in an image.

20. The defect detection method of claim 11, wherein classifying the defects comprises analyzing portions of the at least one of the feature maps and image data containing at least one defect and refraining from analyzing portions of the at least one of the feature maps and image data not containing a defect.
